(19) United States
(12) Patent Application Publication
(10) Pub. No.: US 2008/0014572 A1
(43) Pub. Date: Jan. 17, 2008

(54) Methods and systems for molecular inhibition

(75) Inventors: Edward K.Y. Jung, Bellevue, WA (US); Nathan P. Myhrvold, Medina, WA (US); Lowell L. Wood, Livermore, CA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC, a limited liability corporation of the State of Delaware

(21) Appl. No.: 11/487,180

(22) Filed: Jul. 13, 2006

Related U.S. Application Data
(63) Continuation-in-part of application No. 11/487,133, filed on Jul. 13, 2006.

(51) Int. Cl. C12Q 1/00; G06F 19/00

(52) U.S. Cl. 435/4; 702/19

(57) ABSTRACT

Methods and systems are described which identify the structure of a biochemical or pathogenic molecule as well as at least one interacting molecule structure. These structures may be predicted to form at least one complex. In some embodiments, the stability and toxicity of at least one molecule structure and/or complex may be predicted.

### CROSS-REFERENCE TO RELATED APPLICATIONS

### Related Applications

1. U.S. patent application Ser. No. 11/487,133, entitled METHODS AND SYSTEMS FOR TREATING DISEASE, naming Edward K. Y. Jung and Lowell L. Wood, Jr., as inventors, filed Jul. 13, 2006.

2. U.S. patent application Ser. No. 11/525,758, entitled METHODS AND SYSTEMS FOR MOLECULAR INHIBITION, naming Edward K. Y. Jung, Nathan P. Myhrvold, and Lowell L. Wood, Jr., as inventors, filed Sep. 22, 2006.

3. U.S. patent application Ser. No. 11/525,760, entitled METHODS AND SYSTEMS FOR TREATING DISEASE, naming Edward K. Y. Jung and Lowell L. Wood, Jr., as inventors, filed Sep. 22, 2006.

All subject matter of the Related Applications and of any and all parent, grandparent, great-grandparent, etc. applications of the Related Applications is incorporated herein by reference to the extent such subject matter is not inconsistent herewith.

### SUMMARY

In some aspects, methods comprise: predicting a structural model of a first complex consisting essentially of a biochemical molecule structure and a first interacting molecule structure; in response to the predicted structural model of the first complex, selecting a second interacting molecule structure predicted to associate with the first complex; predicting a structural model of a second complex, consisting essentially of the first complex and the second interacting molecule structure; and in response to the predicted structural model of the second complex, selecting a third interacting molecule structure predicted to associate with the second complex. In some aspects, methods comprise: identifying an interacting molecule structure that is predicted to form a primary complex with a pathogenic molecule structure; predicting the structure of the primary complex formed by the pathogenic molecule structure and the interacting molecule structure; identifying a secondary interacting molecule structure that is predicted to form a secondary complex in association with the primary complex; predicting a structure of the secondary complex; and identifying at least one additional interacting molecule structure predicted to form a tertiary complex in association with the secondary complex. Also included are computer instructions, which, when run on a computing device, cause the computing device to: define a model structure of a pathogenic molecule, identify a first interacting molecule structure predicted to be capable of associating with the pathogenic molecule structure; define a model structure of the pathogenic molecule structure in complex with the first interacting molecule structure; and identify at least two additional interacting molecule structures that are capable of associating with the pathogenic molecule structure simultaneously with the first interacting molecule structure to form an inhibitory complex.

The foregoing summary is illustrative only and is not intended to be in any way limiting. In addition to the illustrative aspects, embodiments, and features described above, further aspects, embodiments, and features will become apparent by reference to the figures and the following detailed description.

### BRIEF DESCRIPTION OF THE FIGURES

FIG. 1 is a diagram, representing steps involved in identifying interacting molecular structures.

FIG. 2 is a diagram, representing steps involved in identifying interacting molecular structures.

FIG. 3 is a diagram, representing steps involved in identifying interacting molecular structures.

FIG. 4 is a diagram representing configurations of molecular structures such as those that may be identified by the methods and systems described herein.

FIG. 5 is a diagram representing configurations of molecular structures such as those that may be identified by the methods and systems described herein.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the spirit or scope of the subject matter presented here.

Those having skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary.

Methods described herein include predicting a structural model of a first complex consisting essentially of a biochemical molecule structure and a first interacting molecule structure; in response to the predicted structural model of the first complex, selecting a second interacting molecule structure predicted to associate with the first complex; predicting a structural model of a second complex, consisting essentially of the first complex and the second interacting molecule structure; and in response to the predicted structural model of the second complex, selecting a third interacting molecule structure predicted to associate with the second complex. Methods described herein also include those identifying an interacting molecule structure that is predicted to form a primary complex with a pathogenic molecule structure; predicting the structure of the primary complex formed by the pathogenic molecule structure and the interacting molecule structure; identifying a secondary interacting molecule structure that is predicted to form a secondary complex in association with the primary complex; predicting a structure of the secondary complex and identifying at least one additional interacting molecule structure predicted to form a tertiary complex in association with the secondary complex. Also described herein are computer instructions which, when run on a computing device, cause the computing device to define a model structure of a pathogenic molecule, identify a first interacting molecule structure predicted to be capable of associating with the pathogenic molecule structure, define a model structure of the pathogenic molecule structure in complex with the first interacting molecule structure, and identify at least two additional interacting molecule structures that are capable of associating with the pathogenic molecule structure simultaneously with the first interacting molecule structure to form an inhibitory complex.

Molecule structures predicted and selected through the methods and systems described herein are thought to be particularly beneficial in regard to applications such as combinatorial chemistry, pharmaceutical discovery, pharmaceutical testing and research, although they are not limited to those embodiments. As used herein, the term “structural model” refers to a model of a structure of a molecule or group of molecules. Similarly, as used herein a “molecule structure” refers to a structural model of a particular molecule or class of molecules. A structural model or molecule structure may include a molecule or molecules in their entirety or it may include only a portion of a molecule or molecules. As used herein a structural model includes, but is not limited to, chemical, atomic and physical models, which may include tertiary structure including one or more atomic coordinates, linear diagrams, space-filling structures or predictions, geometric predictions, structures based on functional groups, structures based on energy states or structures based on chemical or molecular bonds. The structural models contemplated herein may or may not be visually presented and may or may not be represented in a physical form. Structural models may include at least one prediction of the 3-dimensional structure of a molecule or molecules. For example, predicting a structural model of the first complex or the second complex may include a 3-dimensional structure prediction. A structural model may be based entirely or in part on experimentally based data such as nucleic acid or protein sequences, X-ray crystal structures or nuclear magnetic resonance (NMR) data, or the structural model may be based entirely or in part on ab initio predictions. In some embodiments, structural models are based on a combination of experimentally based and predicted techniques. Structural models may be generated by any one of a number of techniques known to those of skill in the art. These include the use of commercially available computer programs such as ChemDraw (sold by Cambridgesoft), HyperChem (sold by Hypercube, Inc.), ICM (sold by MolSoft) and Catalyst (sold by Accelrys), or computer programs that are freeware or shareware such as RasMol (available at http://www.umass.edu/microbio/rasmol/), Protein Explorer (available at http://www.umass.edu/microbio/chime/pe/protexpl/frntdoor.htm) or ArgusLab (available at http://www.planaria-software.com/). In some embodiments, the structural models may already exist in databases such as the publicly accessible Entrez Structure, which is made available through the NCBI (available at http://www.ncbi.nlm.nih.gov/entrez/query.fcgi?db=Structure). In some embodiments, multiple conformations may exist as variants of a structural model, and conformational entropy at physiological or near-physiological conditions may be taken into account when predicting one or more structural models. In some embodiments, multiple related structural models, such as isomers or chiral forms, may be predicted based on the same molecular constituents. As a non-limiting example of methods to predict structural models of proteins, see Kuhlman B. et. al., “Design of a Novel Globular Protein Fold with Atomic-Level Accuracy”, Science 302:1364-1368, (2003), which is herein incorporated by reference. As a non-limiting example of methods to predict structural models of DNA polymerases, see Keller D. and Brozik J., “Framework Model for DNA Polymerases”, Biochemistry 44: 6877-6888 (2005), which is herein incorporated by reference. In some embodiments, multiple methods may be used to determine a structural model, see eg. Nanda V and DeGrado W F, “Automated Use of Mutagenesis Data in Structure Prediction”, Proteins 59:454-466 (2005), which is herein incorporated by reference.

As used herein, a “complex” is a group of molecule structures that are predicted to be capable of association at a molecular level. As used herein, “predicted” may include a purely hypothetical prediction, an analytically derived prediction, structurally identified predictions including computer modeled structures, a prediction based on prior experimental data, a probabilistic assessment, or a combination of these. Predicting a structural model of at least one complex may be performed with electrical circuitry, which may include a processor and/or a memory containing computer instructions. Predicting a structural model of at least one complex may also include accessing information regarding crystal structure and/or retrieving information from a database. In some embodiments, complexes consist of structural models in their entirety while in others complexes include one or more partial structural models. The molecule structures or portions of molecule structures involved in a complex may be predicted to associate by any mechanism, including but not limited to covalent bonding, van der Waals forces, physical force, ionic forces, electrostatic interactions, hydrogen bonds and hydrophobic interactions. In some embodiments, complexes may be predicted by computer software such as ChemDraw (sold by Cambridgesoft), HyperChem (sold by Hypercube, Inc.), ICM (sold by MolSoft), Gaussian (sold by Gaussian, Inc.) and Catalyst (sold by Accelrys). In some embodiments, the complex is based on experimental data such as X-ray crystal structures or NMR data (see for example Istvan, E. S. and Deisenhofer J., “Structural Mechanism for Statin Inhibition of HMG-CoA Reductase”, Science 292:1160-1164, (2001), and Wang C. E., “ConfMatch: Automating Electron-Density Map Interpretation by Matching Conformations”, Acta Crystallographica (Section D) D56: 1591-1611 (2000), which are herein incorporated by reference). Some methods to model peptide structures and the association of peptide structures are also described in U.S. Pat. No. 6,560,542 to Mandell et al. and U.S. Pat. No. 6,865,492 to Mandell et al. In some embodiments, the complex is based on homology with experimentally known interactions (see for example PIP, available at http://www.bmm.icnet.uk/˜pip/). In some embodiments, the structural models are predicted to “associate” together, which as used herein refers to an interaction that has some stability for some time period, although it may be transient. In some embodiments, the complexes are predicted to form by direct association of all of the molecules in the complex while in others some of the associations between molecules in the complex are remote or indirect. Some embodiments may include predicting a structural model of a complex, consisting essentially of a previously predicted complex and an interacting molecule structure. An example is predicting a structural model of a third complex, which consists essentially of the second complex and the third interacting molecule structure. Embodiments include those predicting a structural model of each of N complexes, which consist essentially of the N-1 complex and the N interacting molecule structures. In addition, the selection of each additional interacting molecule structure may be in response to the predicted stability of the interaction between the molecule structures forming the most recently predicted complex.

In some embodiments, it may be desirable to create or obtain molecules corresponding to molecular structures such as those identified, predicted and/or selected herein and to examine the association of those molecules in vitro or in vivo. In addition, in some embodiments it may be desirable to detect the interaction between molecules corresponding to molecular structures identified, predicted and/or selected herein. Multiple methods exist to detect the association between molecules corresponding to molecular structures such as those described herein. Any detection technique known to those of skill in the art or described herein may be used to detect the interaction between molecules corresponding to molecular structures. Detection methods include chemiluminescent, fluorescent or radioactive based techniques as well as those that use ultraviolet, infra-red or visible light. Any method known to those of skill in the art or described herein may be used to test the interaction of molecules corresponding to predicted complexes and associations between molecular structures, including fluorescent quenching, phage display, Fluorescence Resonance Energy Transfer (FRET), Enzyme-Linked Immunosorbent Assay (ELISA), electrophoresis-based methods and polymerase-chain reaction (PCR)-based techniques.

As used herein, “biochemical molecules” are those that are predicted to exhibit at least one biochemical activity in vivo or in vitro in some contexts, including but not limited to activity in physiological or near physiological conditions, activity that involves at least one biological molecule or activity that may occur in a biological system. In some embodiments, the biochemical activity of the molecule is known while in others it is predicted based on factors such as the structure, homology or sequence of a protein or its precursor nucleic acids. Some non-limiting examples of biochemical activities include signal transduction activity, kinase activity, proteinase activity, phosphatase activity, activation, inhibitory activity, methylation activity, acetylation activity, ligation activity, gene transcription alterations, gene expression alterations and induction. Some exemplary methods for the identification and characterization of biological molecules are described in Schneider M., “A Rational Approach to Maximize Success Rate in Target Discovery”, Arch. Pharm. Pharm. Med. Chem. 337:625-633 (2004). In some embodiments, the biochemical molecule structure corresponds to a molecule that is an enzyme. Biochemical molecules with corresponding structures that may be part of the complexes described herein include those that are functional components of retroviruses, virons, viral particles, bacteria, prions, fungi, molds, yeasts, parasites and other biological entities. In some embodiments, there may be a biological molecule associated with other molecules, such as a biological molecule that is a subunit of a larger grouping of molecules. In some embodiments, the structural model of the second complex predicts that the second interacting molecule structure associates with both the biochemical molecule structure and the first interacting molecule structure. In some embodiments, the structural model of the second complex predicts that the second interacting molecule structure directly associates with the biochemical molecule structure while in others the structural model of the second complex predicts that the second interacting molecule structure does not directly associate with the first interacting molecule structure. Depending on the embodiment, the structural model of the third complex may predict that the third interacting molecule structure directly associates with the biochemical molecule structure, the first interacting molecule structure and the second interacting molecule structure simultaneously, and/or it may predict that the third interacting molecule structure directly associates with the biochemical molecule structure or does not directly associate with the second interacting molecule structure. In some embodiments, the biochemical molecule is a “pathogenic” molecule that is known or predicted to have at least one biochemical activity that is disruptive to the normal metabolic stasis of an organism. The pathogenic molecule corresponding to the pathogenic molecule structure may be an enzyme. In some embodiments, the pathogenic molecule is causally associated with a disease state, which includes but is not limited to circumstances where the pathogenic molecule directly causes a disease or is part of a group of causes for a disease. As used herein, “disease state” can encompass not only actual diseases but also metabolic states that are disruptions to normal metabolic stasis, including subnormal metabolic activity, an increased tendency to neoplasia and increased susceptibility to pathogens. While it is contemplated that the methods and systems described herein will be applicable to complexes of molecule structures corresponding to molecules that are suitable for use in the treatment of diseases in humans and other mammals, including domestic and non-domestic animals, the methods and systems described herein are not limited to those applications. Other applications for the methods and systems described herein also include, as non-limiting examples, complexes including plant pathogens, bacteriophages and pathogens affecting non-mammalian animals. Depending on the embodiment, the biochemical molecule structure may correspond to a molecule that is causally associated with a disease state in a human, and/or a disease state in a domestic animal, and/or a disease state in a non-domestic animal. In some embodiments, there is a pathogenic molecule corresponding to the pathogenic molecule structure which is causally associated with a disease state in a human and/or a non-human animal.

As used herein, an “interacting molecule” is a molecule that associates with another molecule or group of molecules in a manner that alters the activity of the group of molecules, is predicted to alter the activity, or is predicted to form a complex in such a manner so as to alter the possibility that other molecules will interact with a known or predicted active site of one or more molecules in the complex. In some embodiments, there is an “interacting molecule structure”, which is the predicted structure of the interacting molecule and may be of any one of a number of types, including but not limited to experimentally-based models, chemical, atomic and physical models, which may include 3-dimensional models, tertiary structure models including one or more atomic coordinates, linear diagrams, space-filling structural predictions, geometric predictions, structures based on functional groups or structures based on chemical bonds. Two or more interacting molecule structures of the same or different types may be predicted to associate with a complex of one or more interacting molecule structures and one or more biologically active molecule structures. In some embodiments, the interacting molecule structures and the complex structure are predicted to associate based on their respective structures and principles of molecular interactions. Although it is anticipated that at least three interacting molecules of the same or different types will be predicted to associate with each biological molecule to form the complex, one of skill in the art will appreciate that the precise number and type of molecules predicted to associate in any complex will depend on a number of parameters present in any given embodiment and may vary over time and in different environmental conditions. Some embodiments include predicting a structural model of a third complex, which consists essentially of the second complex and the third interacting molecule structure. In some embodiments, a series of N additional interacting molecule structures are selected, wherein each interacting molecule structure is predicted to associate with the N-1 complex. Some embodiments include identifying a plurality of additional interacting molecule structures. Embodiments may also include predicting a structural model of a biochemical molecule in complex with a plurality of identified interacting molecule structures. In some embodiments, the tertiary complex is predicted to include more than three interacting molecule structures.

Depending on the embodiment, interacting molecule structures may be selected from a previously identified group of potential interacting molecule structures or any other group of previously identified molecule structures. “Selection” may include the identification of an interacting molecule or interacting molecule structure as appropriate to the embodiment, and may include selection based on desired characteristics of the biochemical molecule structure, interacting molecule or interacting molecule structure such as size, shape, conformation or chemical properties. In some embodiments, selection is made in response to another structure or the characteristics of another structure, including the stability of another structure. Some embodiments may include selecting a series of N additional interacting molecule structures wherein each interacting molecule structure is predicted to associate with the N-1 complex. Selecting a second interacting molecule may include a 3-dimensional structure prediction and/or accessing information regarding crystal structure and/or retrieving information from a database. Selection of a second interacting molecule may also be performed with electrical circuitry, which may include a processor and/or a memory containing computer instructions. As will be recognized by one of skill in the art, the interactions of some molecules or molecular structures may initiate or stabilize a conformational change and therefore additional molecules or molecule structures may be selected in response to this change. In some embodiments, a group of molecules or molecule structures is first identified and then one or more selections are made subsequently. When a group of molecules or molecule structures are identified in advance of selection, the group may be a set of candidate molecules or molecule structures.

In some embodiments, the stability of one or more molecule structures or complexes is predicted. As used herein, “stability” includes stability of the molecular structure, including conformation and chemical composition, within the normal parameters of a given embodiment as well as the predicted constancy of the interactions between the structures within a complex over time or between different environmental conditions. Stability may be predicted by any one of a number of methods, including but not limited to thermal, conformational or chemical predictions or in reference to experimental findings. Complexes may be predicted to be stable over time or they may be predicted to be transitory. Stability may be predicted based on energy minimization methods. In some embodiments, stability is based on the conformational entropy of the molecule or molecules themselves. As will be recognized by a person of skill in the art, molecules and molecular structures are inherently somewhat dynamic depending on the environment and therefore stability may vary overtime and between known or predicted environmental conditions. Depending on the embodiment, stability may be based on thermodynamic predictions, and there may be a range of predicted stabilities at particular temperatures and conditions. Some embodiments include predicting the thermodynamic stability for the structure of at least one complex and may also include identifying at least one interacting molecule structure based on the predicted thermodynamic stability of the structure of at least one complex. In some embodiments, stability is based on predicted metabolic conditions of a given organism, including temperature, metabolic chemistry and the presence or absence of stability-enhancing or stability-decreasing molecules. In some embodiments, the stability of the interaction between the molecule structures forming the first complex and/or the stability of the interaction between molecule structures forming the second complex are predicted. It is also possible to select the second interacting molecule structure in response to the predicted stability of the interaction between the molecule structures forming the first complex, and/or selecting the third interacting molecule structure in response to the predicted stability of the interaction between the molecule structures forming the second complex. In some embodiments, at least one complex is predicted to include an epitope which is recognized by an antibody. In some embodiments, the epitope may be entirely located on a biochemical molecule or an interacting molecule. In other embodiments, the epitope may be formed by the interaction of molecules within a complex. Depending on the structural stability of a complex, epitopes may persist over time or they may be transitory. Epitopes may be predicted based on the structural model of a molecule or complex, or they may be defined by an antibody binding to that epitope. Molecules corresponding to the structures within the primary complex may be predicted to create an epitope that may be recognized by an antibody, and the antibody that binds to the epitope may be identified. Molecules corresponding to the structures within the secondary complex may be predicted to create an epitope that may be recognized by an antibody, and the antibody that binds to the epitope may be identified. At least one interacting molecule structure may be predicted to form an epitope that may be recognized by an antibody, and an antibody that binds to that epitope may be identified.

In some embodiments, the activity of molecules corresponding to one or more molecular structures or complexes is predicted. “Activity” may be a biochemical activity as described above, or it may be a physical or chemical activity that is not limited to biochemical environments. Examples of a physical or chemical activity include thermodynamic stability, the potential to interact with other molecules, radioactivity, chemiluminescence, electron transfer, and magnetic potential. Any activity or alteration in type or level of activity may be part of a prediction. Some embodiments include predicting potential activity of a biological molecule corresponding to the biological molecule structure associated with the first complex and/or predicting potential activity of molecules corresponding to molecular structures in the first, second and/or third complex. Some embodiments include selecting the second interacting molecule structure in response to the predicted activity of molecules corresponding to molecular structures in the first complex, and/or selecting the third interacting molecule structure in response to the predicted activity of molecules corresponding to molecular structures in the second complex. In some embodiments, at least one activity of a pathogenic molecule corresponding to a pathogenic molecule structure is predicted. Pathogenic molecules are involved in a number of biochemical activities, including infection, inflammation, cell lysis, immunosuppression, induction or promotion of neoplasia and breakdown of tissues. In some embodiments, at least one pathogenic molecule is an enzyme, and enzymatic activity may be predicted. In some embodiments, formation of the primary complex is predicted to inhibit activity of the pathogenic molecule corresponding to the pathogenic molecule structure. In some embodiments, the pathogenic molecule corresponding to the pathogenic molecule structure is predicted to have less activity when it is a part of the primary complex than it has when it is not part of the primary complex. In some embodiments, the pathogenic molecule corresponding to the pathogenic molecule structure is predicted to have less activity when it is a part of the secondary complex than it has when it is a part of the primary complex only. In some embodiments, the pathogenic molecule corresponding to the pathogenic molecule structure is predicted to have less activity when it is a part of the tertiary complex than it has when it is part of the secondary complex only. Conformation of the pathogenic molecule structure may be altered by formation of the primary, secondary and/or tertiary complex. Some embodiments include computer instructions which, when run on a computing device, cause the computing device to predict the activity of the pathogenic molecule corresponding to the pathogenic molecule structure and/or predict the activity of molecules corresponding to molecule structures within the inhibitory complex, wherein the additional interacting molecule structures may be identified in reference to predicted activity of molecules corresponding to molecule structures within the inhibitory complex.

In some embodiments, the toxicity of molecules corresponding to molecular structures is predicted. Predictions regarding toxicity may be based on one or a combination of methods, including in vitro or in vivo experimental predictions or structural predictions. Experimental methods to predict toxicity include cell culture testing, mutagenesis assays, teratogenesis assays, LD50 assays and skin irritation assays. Toxicity may also be predicted based on molecular structure or inclusion in a chemical class known to have toxic properties. Toxicity may be predicted to be acute or to occur over time with repeated doses. Toxicity may be predicted based on a molecule acting alone or by the action of a combination of molecules. Some embodiments include identifying a set of candidate interacting molecules that are predicted to not be toxic to a mammal, selecting a first interacting molecule from the identified set of candidate interacting molecules, and predicting the structure of the identified first interacting molecule. Embodiments may also include identifying a set of candidate interacting molecules, predicting the toxicity of the identified candidate interacting molecules and predicting the structure of a group of the identified candidate interacting molecules, as well as selecting identified molecules having a predicted toxicity below a selected level. In some embodiments, interacting molecule structures correspond to molecules that are not predicted to be toxic to a human, and/or not predicted to be toxic to a domestic animal. In some embodiments, a molecule or molecules corresponding to the first interacting molecule structure and/or at least one additional interacting molecule structure are predicted to be nontoxic to a human.

In some embodiments, methods as described herein will be carried out by an individual or group of individuals directing computing devices which perform various aspects of the methods. For example, an individual or group of individuals may operate a computer interface or group of computer interfaces to initiate computing devices to carry out methods as described herein. It is also possible for some portion of the methods as described herein to be carried out outside of a computer system and the remaining portion to be carried out within a computer system. For example, a interacting molecule and/or a pathogenic molecule may be identified through clinical or chemical means, and the remaining interacting molecule(s) and/or pathogenic molecule(s) may be identified and corresponding structures predicted through the use of a computer system. Some embodiments include computer instructions which, when run on a computing device, cause the computing device to carry out a group of steps. In some embodiments, the computer steps are implemented by a data processing system. Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems. An individual or group of individuals may direct computer devices to carry out methods and operate systems as described herein.

Some embodiments include the use of computer instructions that, when run on a computing device, cause the computing device to carry out a series of instructions. Some embodiments include computer-readable media that contains computer instructions which, when run on a computer, cause the computer to perform some of the methods described herein. For example, computer readable media may include computer instructions which, when run on a computer, cause the computer to perform a method comprising: predicting a structural model of a first complex consisting essentially of a biochemical molecule structure and a first interacting molecule structure; in response to the predicted structural model of the first complex, selecting a second interacting molecule structure predicted to associate with the first complex; predicting a structural model of a second complex, consisting essentially of the first complex and the second interacting molecule structure; and in response to the predicted structural model of the second complex, selecting a third interacting molecule structure predicted to associate with the second complex. As a further example, computer readable media may include computer instructions which, when run on a computer, cause the computer to perform a method comprising: identifying an interacting molecule structure that is predicted to form a primary complex with a pathogenic molecule structure; predicting the structure of the primary complex formed by the pathogenic molecule structure and the interacting molecule structure; identifying a secondary interacting molecule structure that is predicted to form a secondary complex in association with the primary complex; predicting a structure of the secondary complex and identifying at least one additional interacting molecule structure predicted to form a tertiary complex in association with the secondary 4complex. In some embodiments, computer instructions may comprise a model structure corresponding to a pathogenic molecule, and the pathogenic molecule may be causally associated with a disease state. The disease state may affect a human, and/or a domestic animal, and/or a non-domestic animal. The pathogenic molecule may be an enzyme. Computer instructions may include that least one interacting molecule structure may be predicted to be nontoxic, including being nontoxic to a human. Computer instructions may include those that cause the computer device to access a database. Computer instructions may include predicting the activity of the pathogenic molecule corresponding to the pathogenic molecule structure, and/or the activity of at least one molecule corresponding to at least one molecule structure within the inhibitory complex. Computer instructions may also include those that cause the computer device to predict a structural model of the pathogenic molecule structure, the first interacting molecule structure and at least two additional interacting molecule structures in association. Computer instructions may also include those that cause the computer device to predict the stability of the predicted structural model at metabolic temperatures and conditions. Predicting the structural model may further include: 3-dimensional modeling, tertiary structure comprising one or more atomic coordinates, accessing information regarding crystal structure and/or accessing a database.

Further aspects of the methods and systems described herein are described in the Figures as discussed below.

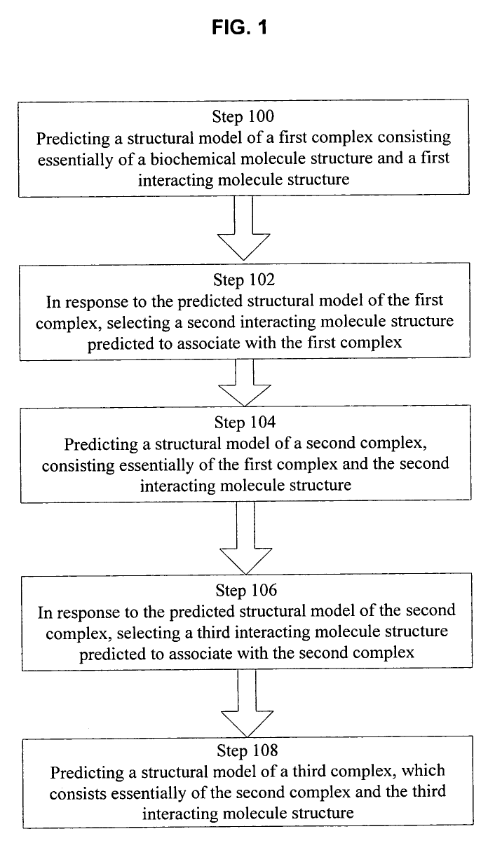

As diagrammed in FIG. 1, an illustrative method begins at Step 100 with predicting a structural model of a first complex consisting essentially of a biochemical molecule structure and a first interacting molecule structure. The first biochemical molecule structure may be identified through experimental analyses, predictive analyses, the additional approaches for prediction described herein or may be received from a separate source. The biochemical molecule structure and the first interacting molecule structure may be identified in any sequence or simultaneously. In one approach identifying the first interacting molecule structure includes identifying one or more interacting molecule structures that are predicted to associate with variable specificity and stability to the first biochemical molecule structure to form one or more respective complexes of the first biochemical molecule. For clarity of presentation, the one or more respective complexes will be referred to subsequently to the first complex. By way of non-limiting examples of binding site prediction and properties and resulting molecular structures, see Istvan E. S. and Deisenhofer J., “Structural Mechanism for Statin Inhibition of HMG-CoA Reductase”, Science 292: 1160-1164, (2001) and Mei Y, Xiang Y, Zhang D W and Zhang J Z H, “Quantum Study of Mutational Effect in Binding of Efavirenz to HIV-1 RT”, Proteins, 59:489-495 (2005), which are herein incorporated by reference.

The method continues in Step 102, including in response to the predicted structural model of the first complex, selecting a second interacting molecule structure predicted to associate with the first complex. The second interacting molecule structure may be identified through experimental analyses, predictive analyses, or additional prediction methods described herein and known in the art. As used in this context, “in response to” includes a selection made based directly on the structural model of the first complex as well as selection(s) made in whole or in part based on the biochemical molecule structure and/or the first interacting molecule structure.

Step 104 includes predicting a structural model of a second complex consisting essentially of the first complex and the second interacting molecule structure. This prediction may be made by any of the methods described herein or known in the art.

Step 106 further describes in response to the predicted structural model of the second complex, selecting a third interacting molecule structure predicted to associate with the second complex. The third interacting molecule structure may be identified through experimental analyses, predictive analyses, or the additional approaches to prediction described herein or known in the art.

Step 108 shows predicting a structural model of a third complex which consists essentially of the second complex and the third interacting molecule structure. This prediction may be made by any of the methods described herein or known in the art.

While the diagrammatic representation of FIG. 1 shows the Steps 100, 102, 104, 106 and 108 sequentially, the order of the steps is not necessarily sequential or as shown. For example, the identification of the interacting molecule structures may be part of, interleaved with, or even responsive to the development of the structural model of the first complex.

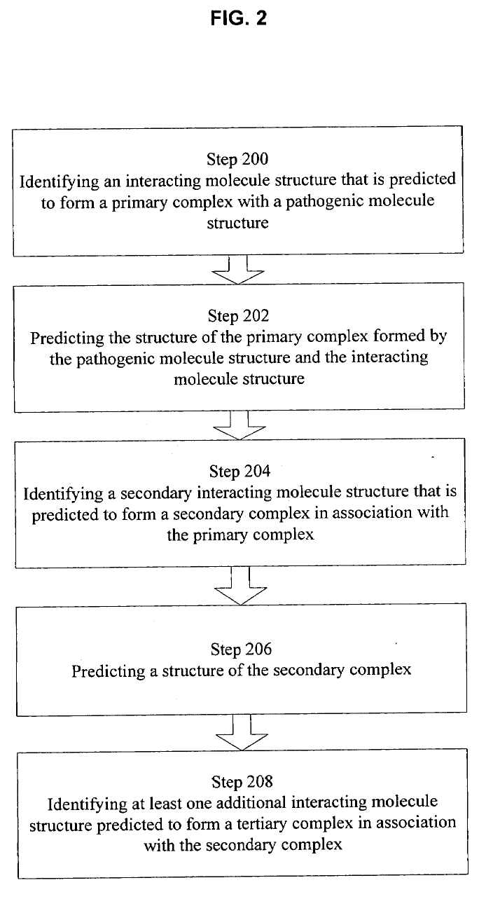

FIG. 2 diagrams steps in other illustrative embodiments, which may be carried out in any order or sequence. Step 200 includes identifying an interacting molecule structure that is predicted to form a primary complex with a pathogenic molecule structure. The pathogenic molecule and the interacting molecule structures may be identified by any method described herein or known in the art, including through experimental analyses, predictive analyses or available databases. In some embodiments, the identification includes the prediction of a number of potential interacting molecule structures and the selection of at least one based on some preferred characteristic, such as thermal stability or chemiluminescent properties.

Step 202 describes predicting the structure of the primary complex formed by the pathogenic molecule structure and the interacting molecule structure. This and all predictions described may be accomplished through any one of a number of methods including those heretofore mentioned and others known in the art. This and all predictions described may take into account one or more environmental conditions expected to be relevant for a given embodiment, such as ambient temperature or pH of a surrounding liquid. The primary complex structure and those of all other complexes described in FIG. 2 may have been previously identified through experimental or predictive analyses.

Step 204 shows identifying a secondary interacting molecule structure that is predicted to form a secondary complex in association with the primary complex. Step 206 further shows predicting a structure of the secondary complex. Depending on the embodiment, multiple structures may be predicted, and one or more may be selected for use in other steps.

Step 208 describes identifying at least one additional interacting molecule structure predicted to form a tertiary complex in association with the secondary complex. Where there are multiple additional interacting molecule structures, they may be identified separately, or in conjunction with each other. Multiple interacting molecule structures may be part of a larger group of potential interacting molecule structures from which at least two are selected.

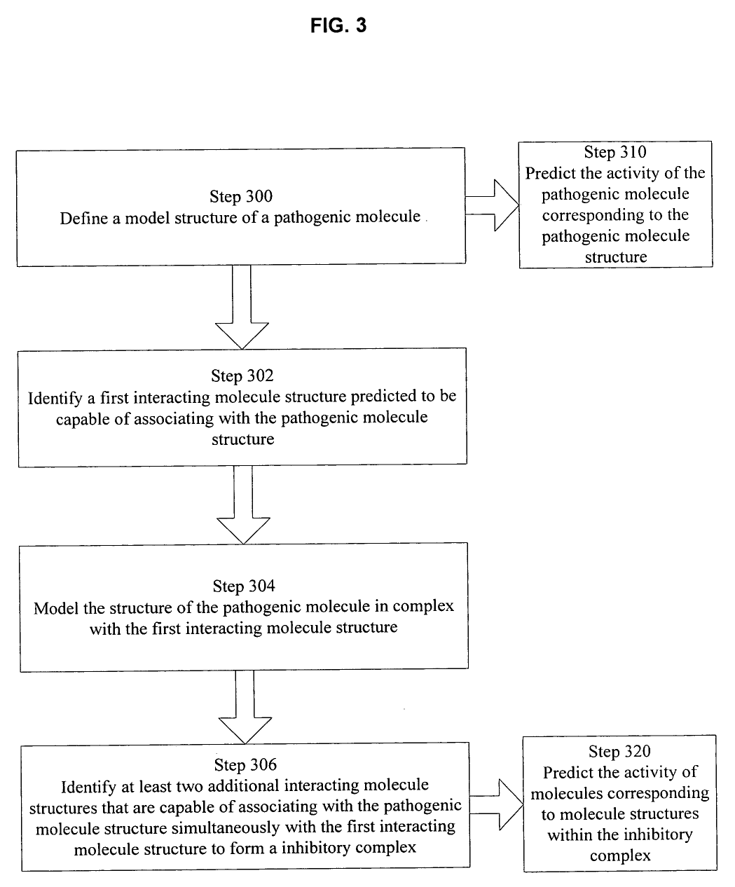

As diagrammed in FIG. 3, some embodiments include computer instructions which, when run on a computing device, cause the computing device to carry out a group of steps. These steps are shown in a sequential order but they need not be carried out in a particular order. For example, an interacting molecule structure may be identified (as in Step 302) before a model structure of a pathogenic molecule is defined (as in Step 300). In some embodiments, the computer steps are included in the memory of a computer device which also may include an input/output (I/O) device and a network connection.

Step 300 describes instructions to define a model structure of a pathogenic molecule. This model structure may be previously generated or it may be generated by any of the methods herein described or known in the art. In some embodiments, the pathogenic molecule structure may be initially selected based on a relevant activity of the pathogenic molecule, such as infectivity, pathogenicity, stability or a biological activity such as those described above.

Step 302 shows instructions to identify a first interacting molecule structure predicted to be capable of associating with the pathogenic molecule structure. This molecule structure may be identified as part of a group of potential interacting molecule structures and then selected from that group, or it may be selected as an individual molecule structure. In some embodiments, more than one first interacting molecule structure may be identified.

Step 304 includes instructions to model the structure of the pathogenic molecule in complex with the first interacting molecule structure. Multiple structures may be modeled, for example for different metabolic or environmental conditions.

Step 306 describes instructions to identify at least two additional interacting molecule structures that are capable of associating with the pathogenic molecule simultaneously with the first interacting molecule structure to form an inhibitory complex. These molecule structures may be selected from a previously defined group, or they may be identified in response to the model generated in Step 304.

Step 310, which shows instructions to predict the activity of the pathogenic molecule corresponding to the pathogenic molecule structure, may be included. Some embodiments also include the instructions of step 320 to predict the activity of molecules corresponding to molecule structures within the inhibitory complex. The activity or activities of molecules corresponding to molecule structures within the inhibitory complex may be a biological activity such as those described above, or it may be a lack of activity such as the lack of pathogenic molecule activity such as that described in Step 310. Instructions shown in Steps 310 and/or 320 may be carried out at any time before, after or interleaved with additional steps, or Steps 310 and/or 320 may be dispensed with in any given embodiment.

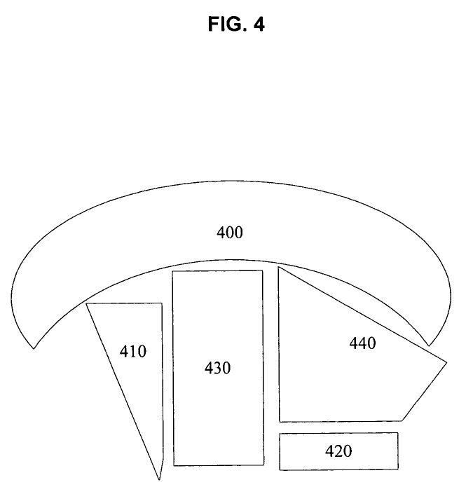

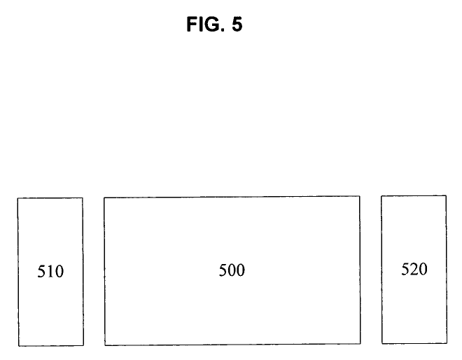

FIGS. 4 and 5 show representative diagrams of some potential configurations of molecular structures such as those that may be identified through the methods and systems described herein.

FIG. 4 shows a potential grouping of molecular structures, including biochemical molecule structure 400. In complex with biochemical molecule structure 400 are interacting molecule structures 410, 420, 430 and 440. As is visually displayed in this Figure, each of the interacting molecule structures need not directly associate with each other or with the biochemical molecule structure. For example, interacting molecule structure 420 associates directly with interacting molecule structures 430 and 440 but not with interacting molecule structure 410 or biochemical molecule structure 400.

FIG. 5 shows an alternative potential grouping of a biochemical molecule structure and interacting molecule structures. Biochemical molecule structure 500 associates with both interacting molecule structures 510 and 520, although structures 510 and 520 do not directly associate with each other. In some embodiments, interacting molecule structures in a arrangement similar to that shown in FIG. 5 would alter the potential of biochemical molecule structure 500 to change conformation, for example to reduce the potential for a predicted binding or active site to be exposed on structure 500.

Further aspects of the methods and systems described herein are illustrated in the Examples discussed below.

### EXAMPLE 1

HMG-CoA (3-hydroxy-3-methylglutaryl-coenzyme A) reductase (HMGR) catalyses the committed step in cholesterol biosynthesis. A group of molecules known as statins are known to associate with HMGR and reduce its activity, thereby decreasing cholesterol biosynthesis.

The molecular structure of HMGR and a group of statin molecules has been described singly and in complex (see Istvan E. S. and Deisenhofer J., “Structural Mechanism for Statin Inhibition of HMG-CoA Reductase”, Science 292: 1160-1164, (2001), which is herein incorporated by reference). Embodiments of the methods described herein are applicable for the identification of other molecular structure(s) that may also associate with one or more of the HMGR-statin complexes. These additional molecular structures may be predicted to alter the stability of a HMGR-statin complex, including increasing the stability of the complex. Methods and systems as described herein will also be applicable to determine the toxicity of the molecular structure(s) identified, both singly as well as in complex.

### EXAMPLE 2

HIV-1 reverse transcriptase (RT) is essential for HIV replication but is not required for normal cell replication. A group of molecules known as nonnucleoside reverse transcriptase inhibitors (NNRTIs) are known to associate with RT and inhibit its activity.

The molecular structure of RT and at least one NNRTI, Efavirenz, have been described singly and in complex (see Mei Y., Xiang Y., Zhang D. W. and Zhang J. Z. H., “Quantum Study of Mutational Effect in Binding of Efavirenz to HIV-1 RT”, Proteins, 59:489-495 (2005), which is herein incorporated by reference). Embodiments of the methods and systems described herein are applicable for the identification of further molecular structures that associate with the RT-Efavirenz complex. Methods and systems as described herein may be used to predict molecular structures that will alter the stability of the RT-Efavirenz complex as well as the toxicity of the molecular structures and complexes. Since HIV in general and the RT gene specifically have a high rate of mutation, the molecular structure for RT will depend on the particular subtype being described in any given situation. In some instances, there will be a group of RT subtypes present with slightly different molecular structures. Correspondingly, there may be multiple groups of molecular structures that would interact with the RT variants.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link, etc.).

In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

While particular aspects of the present subject matter described herein have been shown and described, it will be obvious to those skilled in the art that, based upon the teachings herein, changes and modifications may be made without departing from this subject matter described herein and its broader aspects and, therefore, the appended claims are to encompass within their scope all such changes and modifications as are within the true spirit and scope of this subject matter described herein. Furthermore, it is to be understood that the invention is defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should NOT be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” and/or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense of one having skill in the art would understand the convention (e.g., “a system having at least one of A, B, and C” would include but not be limited to systems that have A alone, B alone, C alone, A and B together, A and C together, B and C together, and/or A, B, and C together). In those instances where a convention analogous to “at least one of A, B, or C, etc.” is used, in general such a construction is intended in the sense of one having skill in the art would understand the convention (e.g., “a system having at least one of A, B, or C” would include but not be limited to systems that have A alone, B alone, C alone, A and B together, A and C together, B and C together, and/or A, B, and C together).

As used herein, the term “about” or “consists essentially of” refers to .±15% of any indicated structure, value, or range. Any numerical ranges recited herein (e.g., concentrations, ratios, percentages, sequences, etc.) are to be understood to include any integer within that range and, where applicable, fractions thereof, such as one tenth and one hundredth of an integer (unless otherwise indicated).

The above referenced technical articles are specifically incorporated herein by reference in their entirety for all that they disclose and teach. In an event of any conflict between the instant application and a referenced technical article, the instant application controls.

Although the methods, devices, systems and approaches herein have been described with reference to certain preferred embodiments, various modifications may be made without deviating from the spirit and scope of the invention. As illustrated by the foregoing examples, various choices of computer modeling programs and experimental techniques may be within the scope of the invention. As has been discussed, the choice of embodiment may depend on the intended application of the system, the environment in which the system is used, cost, personal preference or other factors. Therefore, the full spirit or scope of the invention is defined by the appended claims and their legal equivalent and is not be limited to the specific embodiments described herein

What is claimed is:

1.-80. (canceled)

81. Computer instructions which, when run on a computing device, cause the computing device to:
define a model structure of a pathogenic molecule;
identify a first interacting molecule structure predicted to be capable of associating with the pathogenic molecule structure;
define a model structure of the pathogenic molecule structure in complex with the first interacting molecule structure; and
identify at least two additional interacting molecule structures that are capable of associating with the pathogenic molecule structure simultaneously with the first interacting molecule structure to form an inhibitory complex.

82. The computer instructions of claim 81 wherein the pathogenic molecule is causally associated with a disease state.

83. (canceled)

84. (canceled)

85. (canceled)

86. (canceled)

87. (canceled)

88. The computer instructions of claim 81 comprising instructions causing the computer device to:
predict a structural model of the pathogenic molecule structure, the first interacting molecule structure and at least two additional interacting molecule structures in association.

89. The computer instructions of claim 88 comprising instructions causing the computing device to:
predict the stability of the predicted structural model at metabolic temperatures and conditions.

90. The computer instructions of claim 88 wherein predicting the structural model includes:
3-dimensional modeling.

91. (canceled)

92. The computer instructions of claim 88 wherein predicting the structural model includes:
accessing information regarding a crystal structure.

93. (canceled)

94. The computer instructions of claim 81 comprising instructions causing the computing device to:
predict the activity of the pathogenic molecule corresponding to the pathogenic molecule structure.

95. (canceled)

96. (canceled)

97. (canceled)

98. A computer-readable media containing computer instructions comprising:
one or more instructions for predicting a structural model of a first complex consisting essentially of a biochemical molecule structure and a first interacting molecule structure;
one or more instructions for selecting, in response to the predicted structural model of the first complex, a second interacting molecule structure predicted to associate with the first complex;
one or more instructions for predicting a structural model of a second complex consisting essentially of the first complex and the second interacting molecule structure; and
one or more instructions for selecting, in response to the predicted structural model of the second complex, a third interacting molecule structure predicted to associate with the second complex.

99. The computer-readable media of claim 98, containing computer instructions comprising:
one or more instructions for identifying a set of candidate interacting molecules that are predicted to not be toxic to a mammal;
one or more instructions for selecting a first interacting molecule from the identified set of candidate interacting molecules; and
one or more instructions for predicting a structural model of the identified first interacting module.

100. The computer-readable media of claim 98, containing computer instructions comprising:
one or more instructions for identifying a set of candidate interacting molecules;
one or more instructions for predicting the toxicity of the identified candidate interacting molecules; and
one or more instructions for predicting structural models of a group of the identified candidate interacting molecules.

101. The computer-readable media of claim 100, containing computer instructions comprising:
one or more instructions for selecting identified molecules having a predicted toxicity below a selected level.

102. (canceled)

103. (canceled)

104. The computer-readable medial of claim 98, containing computer instructions comprising:
one or more instructions for predicting potential activity of a biological molecule corresponding to the biological molecule structure associated with the first complex.

105. The computer-readable media of claim 104, containing computer instructions comprising:
one or more instructions for selecting the second interacting molecule structure in response to the predicted potential activity of a biological molecule corresponding to the biological molecule structure associated with the first complex.

106. The computer-readable media of claim 98, containing computer instructions comprising:
one or more instructions for predicting activity of molecules corresponding to molecular structures in the second complex.

107. The computer-readable media of claim 106, containing computer instructions comprising:
one or more instructions for selecting the third interacting molecule structure in response to the predicted activity of molecules corresponding to molecular structures in the second complex.

108. (canceled)

109. The computer-readable media of claim 98, containing computer instructions comprising:
one or more instructions for identifying a plurality of additional interacting molecule structures.

110. The computer readable media of claim 109, containing computer instructions comprising:
one or more instructions for predicting a structural model of a biochemical molecule in complex with a plurality of identified interacting molecule structures.

111. A computer-readable media containing computer instructions which, when run on a computing device, cause the computing device to perform steps comprising:
A. identifying an interacting molecule structure that is predicted to form a primary complex with a pathogenic molecule structure;
B. predicting the structure of the primary complex formed by the pathogenic molecule structure and the interacting molecule structure;
C. identifying a secondary interacting molecule structure that is predicted to form a secondary complex in association with the primary complex;
D. predicting a structure of the secondary complex; and
E. identifying at least one additional interacting molecule structure predicted to form a tertiary complex in association with the secondary complex.

112. The computer-readable media of claim 111, containing computer instructions which, when run on a computing device, cause the computing device to perform steps comprising:
predicting the thermodynamic stability for the structure of the primary complex.

113. The computer-readable media of claim 112, containing computer instructions which, when run on a computing device, cause the computing device to perform steps comprising:
identifying at least one interacting molecule structure based on the predicted thermodynamic stability of the structure of the primary complex.

114. The computer-readable media of claim 111, containing computer instructions which, when run on a computing device, cause the computing device to perform steps comprising:
predicting the thermodynamic stability for the structure of the secondary complex.

115. The computer-readable media of claim 114, containing computer instructions which, when run on a computing device, cause the computing device to perform steps comprising:
identifying at least one interacting molecule structure based on the predicted thermodynamic stability of the structure of the secondary complex.

116. (canceled)

117. The computer-readable of claim 111, wherein molecules corresponding to the structures within the primary complex are predicted to create an epitope that may be recognized by an antibody.

118. (canceled)

119. The computer-readable media of claim 111, wherein molecules corresponding to the structures within the secondary complex are predicted to create an epitope that may be recognized by an antibody.

120. (canceled)

121. The computer-readable media of claim 111, wherein at least one interacting molecule corresponding to an interacting molecule structure is predicted to form an epitope that may be recognized by an antibody.

122. (canceled)
